SEQUENCE LISTING (22 sequences)

SEQ ID NO: 1 - DNA, 2148 bp, Homo sapiens
  gene (1)..(2148): human erythropoietin
atgggggtgcacggtgagtactcgcgggctgggcgctcccgccgcccgggtccctgtttgagcggggatttagcgccccggctattggccaggaggtggctgggttcaaggaccggcgacttgtcaaggaccccggaagggggaggggggtggggcagcctccacgtgccagcggggacttgggggagtccttggggatggcaaaaacctgacctgtgaaggggacacagtttgggggttgaggggaagaaggtttgggggttctgctgtgccagtggagaggaagctgataagctgataacctgggcgctggagccaccacttatctgccagaggggaagcctctgtcacaccaggattgaagtttggccggagaagtggatgctggtagctgggggtggggtgtgcacacggcagcaggattgaatgaaggccagggaggcagcacctgagtgcttgcatggttggggacaggaaggacgagctggggcagagacgtggggatgaaggaagctgtccttccacagccacccttctccctccccgcctgactctcagcctggctatctgttctagaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacggtgagaccccttccccagcacattccacagaactcacgctcagggcttcagggaactcctcccagatccaggaacctggcacttggtttggggtggagttgggaagctagacactgcccccctacataagaataagtctggtggccccaaaccatacctggaaactaggcaaggagcaaagccagcagatcctacggcctgtgggccagggccagagccttcagggacccttgactccccgggctgtgtgcatttcagacgggctgtgctgaacactgcagcttgaatgagaatatcactgtcccagacaccaaagttaatttctatgcctggaagaggatggaggtgagttcctttttttttttttttcctttcttttggagaatctcatttgcgagcctgattttggatgaaagggagaatgatcgggggaaaggtaaaatggagcagcagagatgaggctgcctgggcgcagaggctcacgtctataatcccaggctgagatggccgagatgggagaattgcttgagccctggagtttcagaccaacctaggcagcatagtgagatcccccatctctacaaacatttaaaaaaattagtcaggtgaagtggtgcatggtggtagtcccagatatttggaaggctgaggcgggaggatcgcttgagcccaggaatttgaggctgcagtgagctgtgatcacaccactgcactccagcctcagtgacagagtgaggccctgtctcaaaaaagaaaagaaaaaagaaaaataatgagggctgtatggaatacattcattattcattcactcactcactcactcattcattcattcattcattcaacaagtcttattgcataccttctgtttgctcagcttggtgcttggggctgctgaggggcaggagggagagggtgacatgggtcagctgactcccagagtccactccctgtaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccaggtgagtaggagcggacacttctgcttgccctttctgtaagaaggggagaagggtcttgctaaggagtacaggaactgtccgtattccttccctttctgtggcactgcagcgacctcctgttttctccttggcagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatga

SEQ ID NO: 2 - DNA, 4101 bp, goat
  promoter (1)..(4101): beta-casein promoter
ggatccctcgacctgcaggtcaacggatcacaacaaactggaaaattcttcaagagaagaataccagaccaccctacctgcttcctgagaaatctgtttgctgctcagaagcaacagttagaaccagacatggaacaacagactggttccaaatcaggaaaggagtatgtcaaggctgtatatcgtcaccctgattatttaacttatatgcatagtacataatacaaaatgccaggctggatgaatcgcaagctggaatcaagatttctgggagaaatatcaataaacgagatacaaagatacaccacacttatggcagaaaactaagaagaactaaagagcctcttgatgaaagtgaaagaggagagtgaaaaagccagcttaaaacccaacattcaaaatcaagatcatcatttcatggcaaataaatggggaaacaatggaaacagtgagagactttattttcttgggctccaaaatcactgcagattgtgactacagccatgattaaaagatgcttgctccttggaagagaagctattaccaaactagaaagcatattaaaaagcagagacgttactttgctgactaagttctgtctagtcaaacctatggtttttccagtagtcatatatggatgtgagttgaactataaagaaagctgagcaccaaagaattgatgcttttgaaatttggtgttggagaagtctcttgagagtcccttgaacctgcaaggagatccaaccagtccatcctaaaggaaatcagtcctgaatattcattggaaggactgatgctgaaattgaagattaacgttttggactcacctaatgcagaagagccaactcactagaaaagaccccatgttggcaaaaattgaagccaggaagagaagtgaatgacagaggatgagatggttggatggcatcgttgactgaatggacatgagtctgatcaagttccgggagacagcaaaggacagggctgcctggtctgctgcagtccatggggttgcaaagagtcggtctcaaatgagtaactaaacaacaaccaagcagtagaaaaataaataaaatttgtctctgagatctcagtacctctttctgtgcatatccgtctcctgttattgtactttgtcttctgcttgtaataaagctgtcctgttagtaaaatctgtttgggtcctctgaattcttttagctatcaaaaatggaaggtgattattgtgcaatgtccacctctgagtaatatacagagaataaaagaagggagaaattatgtgcaagttctctctcatctcctgcttctcatttaaaagattctacctcagtgggggctaaaactccacatttaacagtagcaaaaaccaatattccatagcttcttaggaaaccattttttatactcttgtatgtaattacattcaagctcaaaagcaaagaagtgattctgcgttggtgaaggcccaaccatagaaaagaggaagaaaataggccacatactgtgcttcccccatagctcagttggtaaagaatctacctacaatgcaggaggcctgggcttgatccctgggtaagggagatcccctggagaaggaaatggtaacccactccagtactcttgcctgtaaatcccatggacggaggagcctggcagctacagccttggggtggcaagagttggacatgattaacaactaaaccactgccaccactccacatactgagtgctccccagtggcactagtggtaaagaaccacctgccggtgcagaagacattaaagacactggctctatccctgcttgggaagtagggaagatcccctagagagggaaatagcaacccactccagaattcttgcctggaaaatcccatgaatgaagactggcgggctgtagtaactggggtcacaaagagttaaacatgatttagcaactaaacatcaccacattaaaaaaattaccaccaaaatagtcatattccaggctaaggggaataatagcactagtacctgagagaactttctcagattctctgtcaagttcttccttctctcatataaccagtagtctagtttacctcatcagatattaactactcatcgattctaaattatctaattatgggggggggcactacattgcattatattttgtgtccattgactatcactcaatttatttataaaaaattcatccatgttgtttctgtgacagtaactcattcacattaattgtaatatctcattgcattgtatactacaatttatttatacaaaatactattattcacacttctgttgattttaatttggaacatcaacaataacgtggctgagaagcttctttctttagtatattgttaaggatttccttgatcaagattttacctacttttctggtccaattggtgagagacagtcataaggaaatgctgtgtttattgcacaatatgtaaagcatcttcctgagaaaataaaagggaaatgttgaatgggaaggatatgctttcttttgtattccttttctgagaaatcagactttttcaccttggccttggccaccaaaagctaacaaataaaggcatatgaagtagccaaggccttttctagttatatctatgacactgagttcatttcatcatttattttcctgacttcctcctgggtccatatgagcagtcttagaatgaatattagctgaataatccaaatacatagtagatgttgatttgggttttctaagcaatccaagacttgtatgacagtaagatgtattaccatccaacacacatctcagcatgatataaatgcaaggtatattgtgaagaaaaatttttaattatgtcaaagtgcttactttagaaggtcatctatctgtcccaaagctgtgaatatatatattgaaggtaatgaatagatgaagctaaccttgtaaaaatgagtagtgtgaaatacaactacaattatgaacatctgtcactaaagaggcaaagaaacttgaagattgcttttgcaaatgggctcctattaataaaaagtacttttgaggtctggctcagactctattgtagtacttagggtaagaccctcctcctgtatgggctttcattttctttcttgcttccctcatttgcccttccatgaatactagctgataaacattgactataaaagatatgaggccaaacttgagctgtcccattttaataaatctgtataaataatatttgttctacaaaagtattatctaaataaatgttactttctgtcttaaaatccctcaacaaatccccactatctagagaataagattgacattccctggaatcacagcatgctttgtctgccattatctgacccctttctctttctctcttctcacctccatctactcctttttccttgcaattcatgacccagattcactgtttgatttggcttgcatgtgtgtgtgctgagttgcgtctgactgttatcaaccccatgaatgatagtccaccaggctctactgtccatgaaattttccagtcaagaatactggagtggattgcatttcctactccatttgattaatttagtgacttttaaatttctttttccatattcgggagcctattcttcctttttagtctatactctcttcactcttcaggtctaaggtatcatcgtgtgcttgttagcttgttactttctccattatagcttaagcactaacaactgttcaggttggcatgaaattgtgttctttgtgtggcctgtatatttctgttgtgtattagaatttaccccaagatctcaaagacccactgaatactaaagagacctcattgtggttacaataatttggggactgggccaaaacttccgtgcatcccagccaagatctgtagctactggacaatttcatttcctttatcagattgtgagttattcctgttaaaatgctccccagaatttctggggacagaaaaataggaagaattcatttcctaatcatgcagatttctaggaattcaaatccactgttggttttatttcaaaccacaaaattagcatgccattaaatactatatataaacagccactaaatcagatcatt

SEQ ID NO: 3 - DNA, 632 bp, Woodchuck hepatitis B virus
  misc_signal (1)..(632): Woodchuck heptitis virus posttranscriptional regulatory element
accaggttctgttcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtttcgcctcgggctcctcgag

SEQ ID NO: 4 - DNA, 1916 bp, Artificial Sequence
  neomycin resistance gene
gcggccgcgcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtccgatcg

SEQ ID NO: 5 - DNA, 29 bp, Artificial Sequence
  WPRE forward primer
accaggttctgttcctgttaatcaacctc

SEQ ID NO: 6 - DNA, 27 bp, Artificial Sequence
  WPRE reverse primer
ctcgaggagcccgaggcgaaacaggcg

SEQ ID NO: 7 - DNA, 24 bp, Artificial Sequence
  Neomycin resistant gene forward primer
gcggccgcgcgcgtcaggtggcac

SEQ ID NO: 8 - DNA, 29 bp, Artificial Sequence
  Neomycin resistant gene reverse primer
cgatcggacgctcagtggaacgaaaactc

SEQ ID NO: 9 - DNA, 20 bp, Artificial Sequence
  mouse GnT-V Forward primer
cactgttaattcgcccacct

SEQ ID NO: 10 - DNA, 20 bp, Artificial Sequence
  mouse GnT-V Reverse primer
gcttggtcctcctgactctg

SEQ ID NO: 11 - DNA, 20 bp, Artificial Sequence
  CHO GnT-V Forward primer
gtacagagtgacctgccaaa

SEQ ID NO: 12 - DNA, 20 bp, Artificial Sequence
  CHO GnT-V Reverse primer
gtctttgcatagggccactt

SEQ ID NO: 13 - DNA, 20 bp, Artificial Sequence
  mouse GnT-III Forward primer
gctcaggcctctagtaatct

SEQ ID NO: 14 - DNA, 20 bp, Artificial Sequence
  mouse GnT-III Reverse primer
tcctgacccctaacctactc

SEQ ID NO: 15 - DNA, 20 bp, Artificial Sequence
  CHO GnT-III Forward primer
gcatctacttcaarctcgtg

SEQ ID NO: 16 - DNA, 20 bp, Artificial Sequence
  CHO GnT-III Reverse primer
gtgctcrtgggctcccggta

SEQ ID NO: 17 - DNA, 20 bp, Artificial Sequence
  mouse & CHO Fuc-TIV Forward primer
cacacdgtggcccgctacaa

SEQ ID NO: 18 - DNA, 20 bp, Artificial Sequence
  mouse & CHO Fuc-TIV Reverse primer
tcccagaargargtgatgtg

SEQ ID NO: 19 - DNA, 20 bp, Artificial Sequence
  forward primer for transgene check
ctccttggcagaaggaagcc

SEQ ID NO: 20 - DNA, 20 bp, Artificial Sequence
  reverse primer for transgene check
cagccatggaaaggacgtca

SEQ ID NO: 21 - DNA, 20 bp, Artificial Sequence
  hEPO specific forward primer
gtagaagtctggcagggcct

SEQ ID NO: 22 - DNA, 20 bp, Artificial Sequence
  hEPO specific reverse primer
tcatctgtcccctgtcctgc

(19) United States
(12) Patent Application Publication
(10) Pub. No.: US 2010/0205680 A1
(43) Pub. Date: Aug. 12, 2010

(54) MAMMARY GLAND- SPECIFIC HUMAN ERYTHEROPOIETIN EXPRESSION VECTOR, TRANSGENIC ANIMAL AND METHOD FOR PRODUCING HUMAN ERYTHROPOIETIN USING SAME

(76) Inventor: Jin Hoi Kim, Gyeonggi-do (KR)

Correspondence Address: K&L Gates LLP, 3580 Carmel Mountain Road, Suite 200, San Diego, CA, 92130, US

(21) Appl. No.: 12/733,087

(22) PCT Filed: Aug. 8, 2007
(86) PCT No.: PCT/KR2007/003808

§ 371 Date: Feb. 5, 2010

(51) Int. Cl. C12P 21/00; C12N 15/63; C12N 5/07; A01K 67/00

(52) U.S. Cl. 800/7; 435/320.1; 435/325; 800/13; 800/17; 800/18; 800/15; 800/16; 800/14

(57) ABSTRACT

The present invention provides a mammary gland-specific human erythropoietin expression (hEPO) vector, transgenic animal and method for producing human erythropoietin using the same. The inventive hEPO-expressing transgenic animals express a mammary gland-specific EPO at an extremely higher concentration than the convention method. The hEPO produced from inventive transgenic animals shows better stability and superior physiological activity than those of the same kind of commerally available protein. Therefore, the inventive hEPO-expressing transgenic animals can be effectively used for production of EPO showing a superior physiological activity than the existing EPO.

The present invention related to a mammary gland-specific human erythropoietin expression vector, transgenic animal and method for producing human erythropoietin using same.

### BACKGROUND ART

Synthesized in the kidney and to a minor degree in the liver, erythropoietin (EPO) is a glycoprotein hormone as the primary regulator of human erythropoiesis.

EPO has a molecular weight of approximately 34000 to 38000 daltons. Three N-linked sugars are present at asparagines 24, 38, and 83, and one O-linked sugar is present at serine 126.

Since EPO is indispensable to the formation of red blood cells, it has been synthetically produced for use in persons with certain types of anemia—such as due to renal failure, anemia secondary to AZT treatment of AIDS, and anemia associated with cancer.

As EPO is available as a therapeutic agent, its mass production has been sought by recombinant DNA technology in mammalian cell culture. Using cell culture technology, large-scale culture of EPO requires higher production cost with more professional knowledge.

Furthermore, since it is not possible to completely isolate a freshly produced EPO from an animal EPO contained in the culture medium, the purity of a finally produced EPO has low purity and activity. As a result, the recombinant human EPO (hEPO) using the above method has low physiological activity compared to native EPO protein due to different glycosylation.

In contrast, the attraction of using transgenic animals to produce recombinant proteins in body fluids lies in the easier method of separating and purifying the target proteins as compared with the conventional cell culture technology with better activity in a long period of time.

Up to now, expression levels of the target protein in the mammary gland tissue of transgenic animals have been higher than any others.

However, the protein production efficiency in transgenic animals with mammary gland-specific promoters reported thus far is still at a very low level. Furthermore, ectopic expression of a gene by mammary gland-specific vector has been reported.

The Korean Unexamined Publication No. 2001-81456 discloses WAP promoter separated by mammary gland in mice, EPO transgenic expression vector containing hEPO genome gene and transgenic pig.

The Korean Unexamined Publication No. 10-2004-101793 also discloses a transgenic cloned cow to express hEPO in mammary gland, using bovine beta-casein promoter.

Notwithstanding this, the commercialization of hEPO in these transgenic animals has not been reported with any record describing the results of hEPO concentrations in milk and its activity.

To consummate the present invention, the inventor et al. constructed a vector to express a mammary gland-specific hEPO at high efficiency and as a result, transgenic animals containing the expression vector are able to produce hEPO with a higher physiological activity.

### DISCLOSURE OF THE INVENTION

It is an object of the present invention to provide a vector that can express mammary gland-specific hEPO proteins.

It is another object of the present invention to provide the vector-inserted somatic cell in transgenic animals.

It is another object of the present invention to provide the vector-inserted transgenic animals.

It is another object of the present invention to provide a method of producing transgenic animals using the vector.

It is another object of the present invention to provide a method of producing hEPO in transgenic animals.

### Technical Solution

In one embodiment, the present invention provides a vector expressing a mammary gland-specific hEPO protein.

In another embodiment, the present invention provides a pBC1-hEPO-WPRE vector having the DNA sequence identified as the sequence of encoding the beta-casein promoter, a mammary gland-specific promoter; the DNA sequence of encoding hEPO at the 3′-end of the promoter, and the DNA sequence of encoding WPRE.

According to the inventive pBC1-hEPO-WPRE vector, the DNA sequence encoding hEPO may, but not limited to, have the sequence identified as SEQ ID NO:1.

According to the inventive pBC1-hEPO-WPRE vector, the DNA sequence encoding the beta-casein promoter may, but not limited to, have the sequence identified as SEQ ID NO:2.

According to the inventive pBC1-hEPO-WPRE vector, the DNA sequence encoding WPRE may, but not limited to, have the sequence identified as SEQ ID NO:3.

According to the present invention, the aforementioned DNA sequence encoding the beta-casein promoter, hEPO and WPRE include their equivalents which have one more deletions, substitutions, insertions or additions in the DNA sequence.

More specifically, the present invention provides a pBC1-hEPO expression vector which can express mammary gland-specific EPO by inserting hEPO gene (SEQ ID NO:1) to pBC1 expression vector containing goat beta-casein promoter.

If necessary, the inventive expression vector pBC1-hEPO may additionally contain an insulator, WPRE (woodchuck hepatitis virus post-transcriptional regulatory element) or neomycin-resistant gene, thus making it easier to construct the transgenic cell line, maximize the expression amount of target protein and ensure the stability of expression level.

The insulator is a factor that promotes not only the effect of a regulator adjacent to the promoter but also help position-independent expression. It allows a target protein to be stably expressed under the regulation of the beta-casein promoter.

The WPRE is a regulator that may increase the synthesis of a target protein by contributing to the stabilization of mRNA. It allows the target protein to be expressed at large amounts under the regulation of the beta-casein promoter. The WPRE has a base sequence of SEQ ID NO:3.

The neomycin-resistant gene is a gene that shows the resistance to a G418 reagent involved in the cell line establishment. It can act as an efficient selective marker in the establishment of an animal cell line, which express a target protein under the regulation of the beta-casein promoter. The neomycin-resistant gene has a base sequence of SEQ ID NO:4.

The present invention provides 1) WPRE-containing pBC1-hEPO-WPRE vector, and 2) pBC1/hEPO/NEO vector in which the pBC1-hEPO-WPRE vector contains neomycin-resistant gene, as preferred examples of the expression vector which additionally contains these regulators.

These vectors are produced by inserting the WPRE into the 3′ end of the EPO gene within inventive pBC1-hEPO vector, and then inserting the neomycin-resistant gene into the pBC1-hEPO-WPRE vector.

The inventive expression vector pBC1/hEPO/NEO was deposited to the Korea Research Institute of Bioscience and Biotechnology's Biology Resource Center under the accession number KCTC 11159BP on Jul. 26, 2007.

The inventive expression vector may contain, if necessary, another regulators, such as a promoter, an enhancer, a selective marker gene, untranslated region (5′-UTR), 3′-UTR, a polyadenylation signal, a ribosome-binding sequence, a base sequence that can be inserted into a given location of a genome, or intron at its locations.

The present invention also provides a transgenic somatic cell using the expression vector.

In one embodiment of the present invention, the somatic cells of the transgenic pig were deposited to the Biology Resource Center under the accession number KCTC 11160BP on Jul. 26, 2007.

In another embodiment, the present invention provides a fertilized ovum to be collected from nuclear transplantation of transgenic somatic cell using the expression vector to enucleated egg.

The present invention also provides a transgenic animal using the expression vector.

The lactating animals (e.g., pig, mouse, cow, goat, sheep, horse and dog) can be transformed with the expression vector of the present invention.

A method for the production of a transgenic animal using the inventive expression vector is based on the conventional method.

For example, if a mouse is intended for use as a transgenic animal, the production of such animal is conducted as follows:

A fertilized egg is collected from a healthy mouse, and the inventive expression vector is introduced into the fertilized egg. Then, a pseudopregnant mouse is obtained using a vasoligated mouse, and the fertilized egg is implanted into the oviduct of the pseudopregnant mouse as a surrogate mother. Then, transgenic animals among the descendants obtained from the surrogate mother are screened.

If a pig is intended for a transgenic animal, follicular oocytes of healthy gilts are collected and cultured in an in vitro maturation culture medium. After the inventive expression vector is inserted to the collected, cultured somatic cells of a donor, vector-inserted somatic cells are selected for culture. Nuclei are removed from in vitro matured ova to insert the space with donor cells, followed by electric fusing to fuse donor cells and cytoplasm of ova in the completed nuclear transplantation for in vitro culture. After the copied fertilized egg is inserted to a super ovulation-induced fertilized pig, descendants are selected from of transgenic gilts.

Thereafter, an appropriate amount of milk from the transgenic pig is collected to separate and purify a target protein for production of useful proteins (A. Gokana, J. J. Winchenn, A. Ben-Ghanem, A. Ahaded, J. P. Cartron, P. Lambin (1997) Chromatographic separation of recombinant human erythropoietin isoforms, Journal of chromatography, 791, 109-118).

In another embodiment, the present invention provides the production method of hEPO comprising a method of separating and purifying EPO expressed in the milk of transgenic animals.

The conventional processes, including filtration or chromatograph, may be employed for separating and purifying hEPO.

The inventive transgenic animals can express a mammary gland-specific EPO at an extremely higher concentration than the convention method. Specifically, these animals express the target protein in alveolar cells only during lactation.

For example, a transgenic mouse by the inventive expression vector pBC1/hEPO/NEO shows a high EPO expression level of 200,000 to 400,000 IU/mL. Despite the fact that the expression of EPO is not easy due to early death of an embryo, the expression level of EPO in inventive transgenic animal can be achieved up to 1,000 times higher than that found in milk of a transgenic animal using the existing mammary gland-specific promoter.

Further, the EPO produced from inventive transgenic animals shows better stability and superior physiological activity than those of the same kind of commercially available protein.

For example, the EPO produced from a mouse transformed with inventive expression vector pBC1/hEPO/NEO contains a great amount of sialic acid, and exhibits a superior protein activity by acting on the precursor of red blood cells.

Further, EPO produced from inventive transgenic animals increments the levels of platelet, erythrocyte, hemoglobin and hematocrit in the blood during administration.

Therefore, the inventive EPO-expressing vectors and the transgenic animals can be effectively used for production of EPO showing a superior physiological activity than the existing EPO.

### BRIEF DESCRIPTION OF DRAWINGS

FIG. 1 shows the structure of pBC1/hEPO/NEO expression vector of the present invention.

FIG. 2 shows the structure of pBC1/hEPO/NEO expression vector of the present invention.

FIG. 3 shows the PCR results confirming structural elements of a pBC1/hEPO/NEO expression vector of FIG. 2

FIG. 4 shows the expression level in a mouse gene involved in the glycosylation process of mammary gland, liver, and CHO cells.

FIG. 5 shows the expression level of hEPO in a mouse transformed with hEPO expression vector of the present invention.

FIG. 6 shows the expression level of hEPO in a mammary gland of the transgenic mouse of the present invention.

FIG. 7 is the analytical results of hEPO produced from the milk of the transgenic mouse of the present invention.

FIG. 8 shows hEPO produced from the milk of transgenic mouse of the present invention and a two-dimensional electrophoresis gel of the serum in patients with renal failure.

FIG. 9 shows hEPO produced from the milk of transgenic mouse of the present invention, including the analytical results of oligosaccharides of epoetin alpha.

FIG. 10 shows in vitro activity measurements of hEPO produced from the milk of the transgenic mouse of the present invention.

FIG. 11 shows in vivo activity measurements of hEPO produced from the milk of the transgenic mouse of the present invention.

### BEST MODE FOR CARRYING OUT THE INVENTION

This invention will now be described by reference to the following examples which are merely illustrative and which are not to be construed as a limitation of the scope of this invention.

### Example 1

### Production of Optimal Vectors to Express a Mammary Gland-Specific hEPO According to the Present Invention

Under the present invention, optimal vectors secreting hEPO in a mammary gland was produced.

1) Production of pBC1-hEPo Vector

To produce the mammary gland-specific expression vector of the present invention, pBC 1 with goat beta-casein promoter (Invitrogen) was employed so that hEPO genomic DNA (SEQ ID NO: 1) was cloned at its restriction enzyme XhoI position.

2) Production of pBC1-hEPO-WPRE Vector

To increase the expression amount of hEPO, WPRE (woodchuck hepatitis virus post-transcriptional regulatory element) gene was inserted to the pBC1-hEPO vector. WPRE has been shown to play a role as a modulator to ensure better stabilization of mRNA and larger production of finally synthesized proteins.

To ensure that both EPO and mRNA could be transcribed concurrently, WPRE was connected immediately behind hEPO and then, the modulator was inserted to XhoI, a restriction enzyme, from the pBC1-hEPO vector.

Using a forward primer 5′-ACCAGGTTCTGTTCCTGTTAATCAACCTC-3′ (SEQ ID NO:5) and a reverse primer 5′-CTCGAGGAGCCCGAGGCGAAACAGGCG-3′ (SEQ ID NO:6), WPRE was obtained by amplifying 0.6 kb PCR (polymerase chain reaction) product from hepatitis virus and then, it was cloned in pGEM T-easy vector. The 0.6 kb WPRE was cleaved using restriction enzymes (SalI, XhoI) to prepare for the insert site. The pBC1-hEPO cleaved by Xho1 was ligated to a previously arranged vector to produce pBC1-hEPO-WPRE vector (23975 bp).

3) Production of pBC1/hEPO/NEO Vector

To effectively select cells with vectors that could maximize the production of hEPO under the regulation of a mammary gland-specific beta-casein promoter, neomycin-resistant gene was cloned.

Neomycin-resistant gene, which may be resistant to G418 drug, was obtained by amplifying 1.9 kb PCR product using a forward primer 5′-GCGGCCGCGCGCGTCAGGTGGCAC-3′ (SEQ ID NO:7) and a reverse primer 5′-CGATCGGACGCTCAGTGGAACGAAAACTC-3′ (SEQ ID NO:8) from pEGFP-N1 vector (Clontech, Catalog #6085-1) and then, it was cloned in pGEM T-easy vector.

1.9 kb neomycin-resistance gene cloned in T-vector was cleaved by restriction enzymes (NotI, PvuI) to prepare for the insert site. Further, the ampicillin-resistant gene site of the pBC1-hEPO-WPRE vector was cleaved by restriction enzymes (NotI, PvuI) to prepare for the vector site.

Through ligation of the aforementioned insert fragments and vector site, WPRE and neomycin-resistant gene were inserted in the pBC1-hEPO vector to produce pBC1/hEPO/NEO vector.

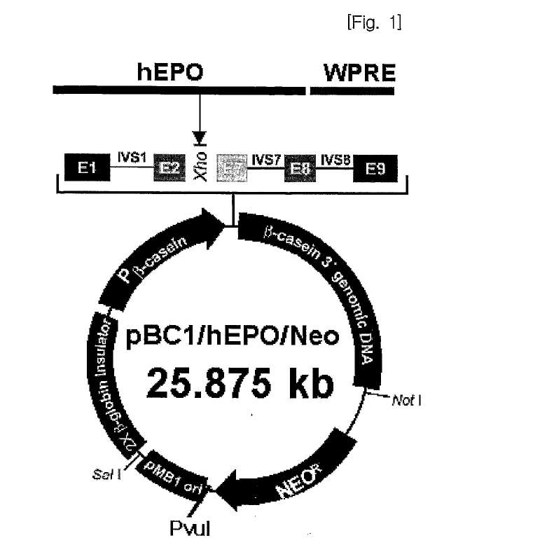

The structure of the inventive expression vector, so obtained, was shown in FIG. 1. hEPO was ultimately expressed under the regulation of beta-casein promoter, a mammary gland-specific promoter.

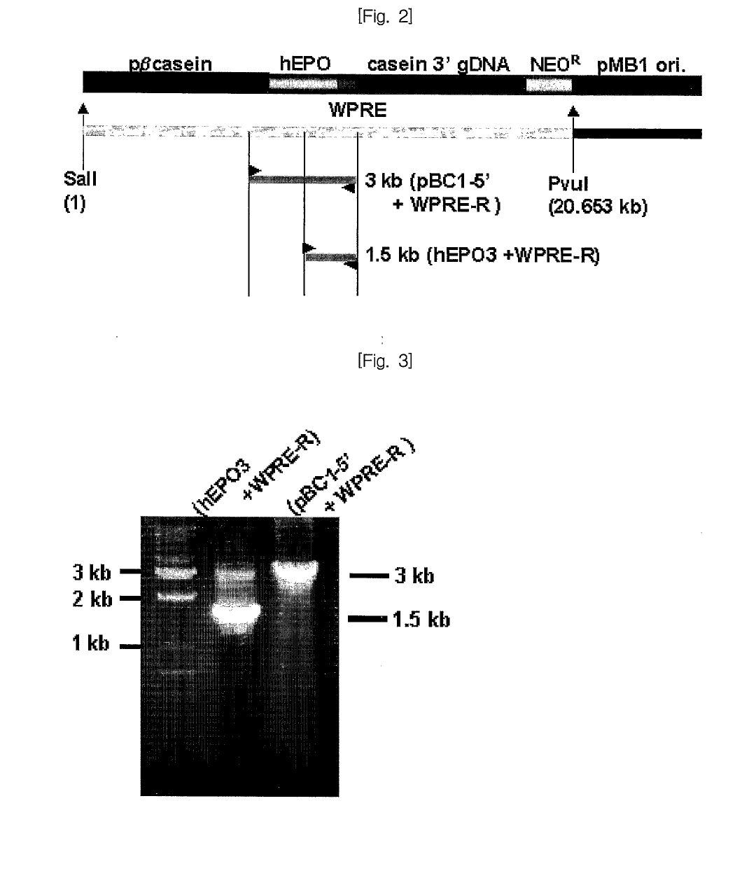

The inventor et al. were able to confirm the proper production of pBC1/hEPO/NEO vector as 3 kb and 1.5 kb of PCR products were obtained through PCR of pBC1-5′+WPRE-R primer pair and hEP03+WPRE-R primer pair (FIGS. 2 and 3).

The inventive expression vector pBC1/hEPO/NEO was deposited to the Korean Research Institute of Bioscience and Biotechnology under the accession number of KCTC 11159BP on July 26, 2007.

With introduction of WPRE modulator, the expression vector pBC1/hEPO/NEO can be used to maximize the expression level of EPO. Further, the neomycin-resistant gene can act as an efficient selective marker in the establishment of an animal cell line.

### Example 2

### Production of hEPO-Expressing Transgenic Animals and Analysis of Expressed hEPO

The following experiments were conducted to confirm the physiological activity and stability profile of hEPO expressed by the mammary gland-specific hEPO-expressing transgenic mouse of this invention.

1) The Expression Profile Analysis of the Gene Involved in Glycosylation in the Mammary Gland of Mouse.

Glycosylation has been shown to play a major role in enhancing protein function as part of a post-translational modification. Different expression profiles of glycosylation have been noted in the prokaryotes (including Escherichia coli), yeasts, animal cells and mice.

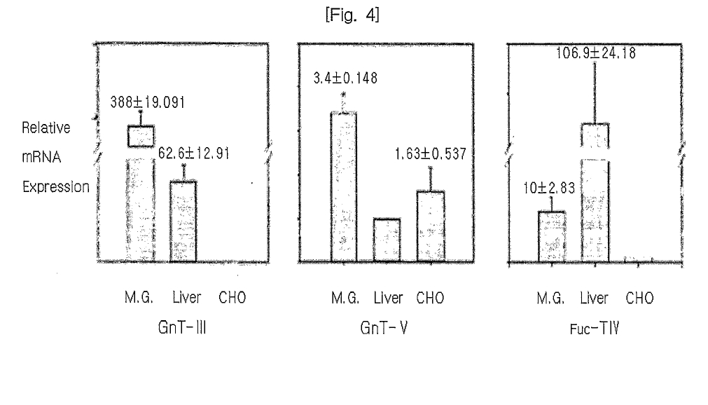

The degree of glycosylation produced in experimental animals undergoing the similar evolution stage to human beings is reported to be similar to that of human body. As a result, the expression of glycosyltransferase involved in the glycosylation of recombinant hEPO in in vivo study was identified in mammary gland and liver of mice, and in CHO cell line, which has been effectively used in the production of EPO (FIG. 4).

Initially, after separation of total RNA from mammary gland, liver and CHO cell line in mice, RT-PCR (reverse transcriptase-polymerase chain reaction) was performed using the primer pairs (table 1) of 3 kinds of glycosyltransferase (GnT-V, GnT-III Fuc-TIV).

TABLE 1

| | | |
|---|---|---|
| Category | Sequence of primer | SEQ ID NO |

| | | |
|---|---|---|
| Mouse GnT-V forward primer | 5′-CACTGTTAATTCGCCCACCT-3′ | 9 |
| Mouse GnT-V reverse primer | 5′-GCTTGGTCCTCCTGACTCTG-3′ | 10 |
| CHO GnT-V forward primer | 5′-GTACAGAGTGACCTGCCAAA-3′ | 11 |
| CHO GnT-V reverse primer | 5′-GTCTTTGCATAGGGCCACTT-3′ | 12 |
| Mouse GnT-III forward primer | 5′-GCTCAGGCCTCTAGTAATCT-3′ | 13 |
| Mouse GnT-III reverse primer | 5′-TCCTGACCCCTAACCTACTC-3′ | 14 |
| CHO GnT-III forward primer | 5′-GCATCTACTTCAARCTCGTG-3′ | 15 |
| CHO GnT-III reverse primer | 5′-GTGCTCRTGGGCTCCCGGTA-3′ | 16 |
| Mouse & CHO Fuc-TIV forward primer | 5′-CACACDGTGGCCCGCTACAA-3′ | 17 |
| Mouse & CHO Fuc-TIV reverse primer | 5′-TCCCAGAARGARGTGATGTG-3′ | 18 |

The test results indicated that as shown in FIG. 4, two kinds of glycosyltransferase (GnT-III and GnT-V) were highly expressed in the mammary gland, compared to liver and CHO cell line.

The same trend was detected in Fuc-T having more expression in the mammary gland than CHO cell line. It demonstrates that recombinant hEPO derived from the mammary gland is superior to the currently available EPO products of CHO cell line in terms of physiological activity.

2) Production of Transgenic Mouse

Under the regulation of mammary gland-specific beta-casein promoter constructed in the Example 1, a transgenic mouse was produced by microscopic injection in the presence of 23 kb pBC1/hEPO/NEO expression vector (FIG. 1) expressing EPO (Macrogen Co. in Korea).

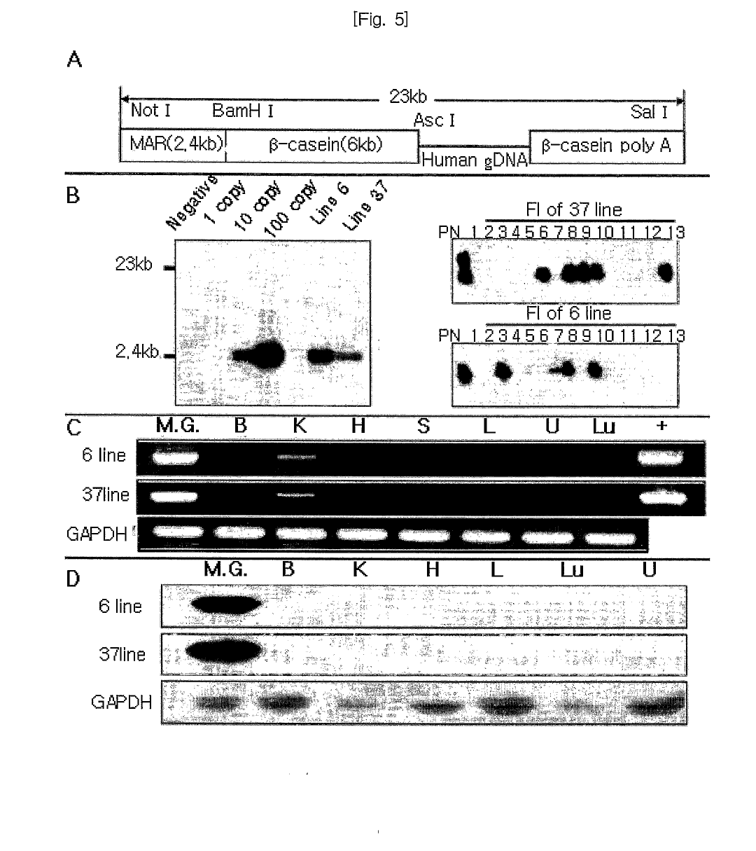

We used a transgenic mouse BDF1 (C587BL/6 DBA). After injecting the pBC1/hEPO/NEO to 738 fertilized eggs, 700 eggs were transplanted to 30 surrogate mothers. From a total of 85 living mice born after several weeks, 9 transgenic mice were identified by PCR and Genomic Southern blot (Table 2 and FIG. 5B).

The PCR was performed using forward primers (5′-CTCCTTGGCAGAAGGAAGCC-3′; SEQ ID NO:19) and reverse primers (5′-CAGCCATGGAAAGGACGTCA-3′; SEQ ID NO:20) for checking TG. The resulting PCR produce size was estimated to be 600 bp.

Southern blot was performed using hEPO genomic DNA as a probe (total 2.3 kb gene including SEQ ID NO:1).

FIG. 5B shows the results of PCR and Southern blot covering the EPO gene identified line 6 and line 37, two representative lines in a transgenic mouse expressing the mammary gland-specific EPO. Based on the results of FIG. 5B, it was noted that 10 to 30 copies of EPO gene are inserted into the chromosomes and inherited to the next generation through reproductive cells.

TABLE 2

| | No. of | No. of | | No. of | No. of |
|---|---|---|---|---|---|
| | embryos | embryos | No. of | mice | transgenic |
| Mouse strain | injected | transferred | recipients | born | mice (%) |
| BDF1 | 738 | 700 | 30 | 85 | 9 (10.6) |
| (C57BL/6 × | | | | | |
| DBA) | | | | | |

3) Identification of Mammary Gland-Specific EPO in the Transgenic Mouse

From the inventive transgenic mouse, we identified whether the mammary gland-specific EPO gene would be expressed.

Each RNA from various tissues of the transgenic mouse in lines 6 and 37, such as mammary gland, brain, kidney, heart, spleen, liver, uterus and lung, was extracted. Then the RT-PCR and Northern blot analysis were performed using a common method that has been known in the prior art.

To detect EPO cDNA, RT-PCR reaction was performed using a hEPO-specific forward primer (SEQ ID NO:21) and a hEPO-specific reverse primer (SEQ ID NO:22).

| | |
|---|---|
| 5′-GTAGAAGTCTGGCAGGGCCT-3′ | (SEQ ID NO: 21) |
| 5′-TCATCTGTCCCCTGTCCTGC-3′ | (SEQ ID NO: 22) |

Northern blot analysis was performed using EPO full genomic DNA as a 2.3 kb probe, which was the same probe as used in Southern blot analysis.

From FIG. 5C, the results of RT-PCR showed that a higher level of EPO was observed at the mammary gland, compared with extremely low level at the kidney. As shown in FIG. 5D, the results of Northern blot analysis revealed that the EPO gene was specifically expressed in the mammary gland, while it was not observed in any other tissues (In the drawings, B: brain, H: heart, S: spleen, L: liver, U: uterus and Lu: lung).

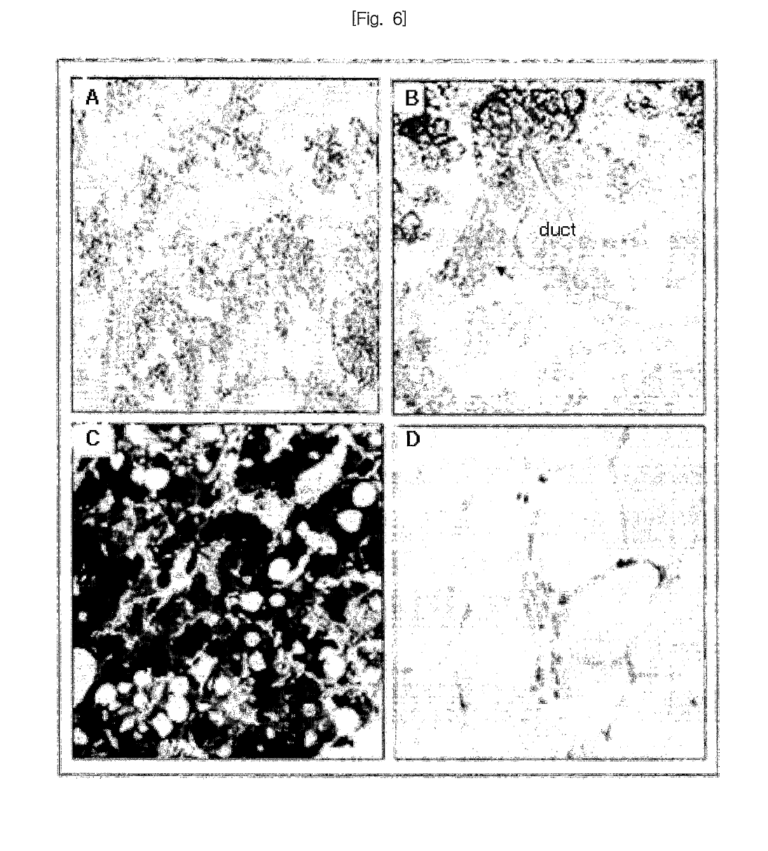

To examine the expression of EPO protein and expressed cells, immunohistochemistry was performed on various tissues of the transgenic animal using anti-human antibody (AB-286-NA, R&D system) as EPO antibody (FIG. 6). The EPO levels were not identified in the mammary gland of control mouse in Day 16 gestation, as shown in FIG. 6A. By contrast, the EPO levels could be identified in the mammary-gland alveolar cell of the transgenic mouse in Day 16 gestation (FIG. 6B) and 5 days after delivery (FIG. 6C). In Day 16 after delivery (FIG. 6D), the expression levels of EPO were not detected from the degenerated alveolar cell of mammary gland.

As shown in FIG. 6, the EPO levels were specifically detected in the alveolar cells of mammary gland during lactation in hEPO transgenic mouse of the present invention

### 4) Analysis of EPO Levels and Stability Profile

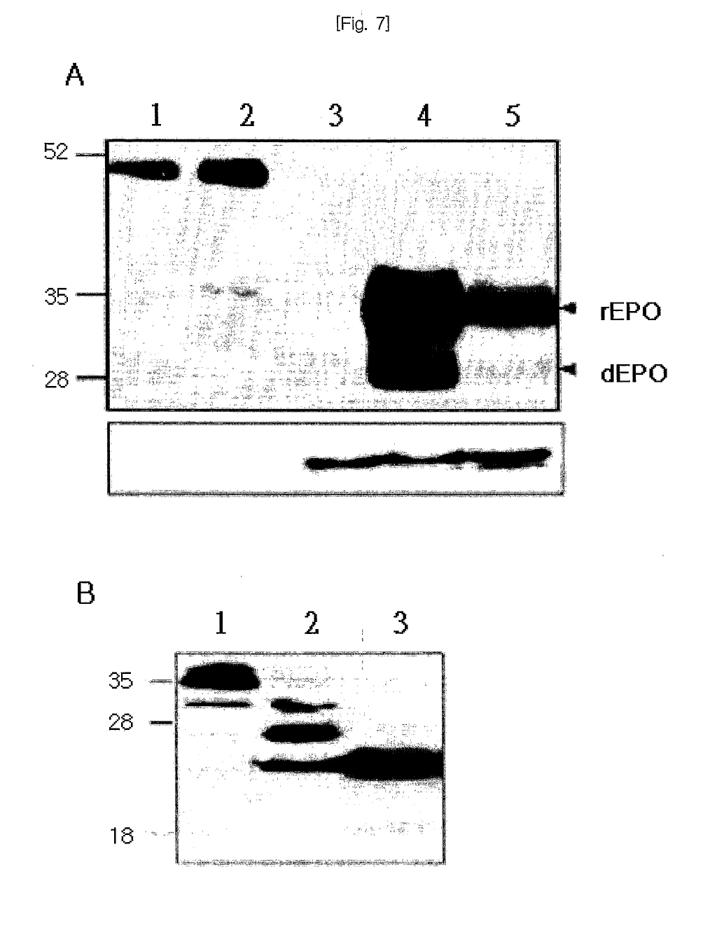

To investigate the EPO levels contained in the milk of mammary gland in the transgenic mouse, the conventional Western blot method was performed using anti-human antibody (AB-286-NA, R&D system) as EPO antibody, as shown in FIG. 7.

FIG. 7A is the results of Western blot on the milk of transgenic mouse; lane 1 and 2 represent GST-tagged EPO antibodies (5 ng and 10 ng) in positive control; lane 3 represents milk of a control mouse group; lane 4 and 5 represent the EPO levels in the milk of each transgenic mouse in lines 6 and 37, respectively.

As shown in FIG. 7A, we identified a protein with its molecular mass of 34 KDa in the milk of inventive transgenic mouse, comprising the range of 0.7 to 1.4% in total proteins.

To confirm the glycosylation patterns of EPO derived from the milk of transgenic mouse, Western blotting was performed using N-glycosidase F or N-glycosidase-F and O-glycosidase.

From FIG. 7B, lane 1 is a milk-derived EPO; lane 2 is a fragment of EPO digestion by N-glycosidase-F, and lane 3 is a fragment of EPO digested by N-glycosidase-F and O-glycosidase.

FIG. 7B shows that the EPO with its molecular mass of 18 KDa was finally identified as three N-glycosylation sites and O-glycosylation site were digested, demonstrating the proper post-modification.

To confirm the EPO levels in the milk of hEPO transgenic mouse, quantitative assay was performed using ELISA kit (Human Erythropoietin ELISA, #01630, Stem Cell Technology). The EPO levels were in the range of 200,000 to 400,000 IU/mL during a lactation period of 1 to 5 days (Table 3)

TABLE 3
Quantitative analysis of EPO in the milk of transgenic mouse

| | Mice | ELISA kit |
|---|---|---|
| Line | Days of lactation | (Stem cell technology) |
| 6 | 1 | 456 ± 33 IU/μL |
| 6 | 3 | 383 ± 28 IU/μL transgenic |
| 6 | 5 | 233 ± 24 IU/μL |
| 37 | 3 | 384 IU/μL |

*rhEPO concentration was measured after complete N- and O-digestion (18 kDa).

To confirm the stability of EPO, two-dimensional analysis was performed using hEPO detected in the milk of transgenic mouse and blood samples in patients with renal failure.

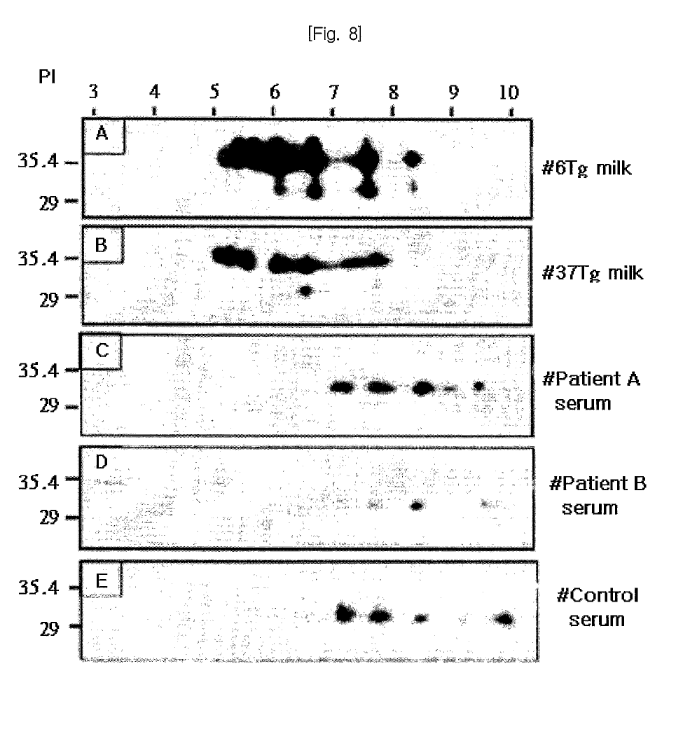

From the two dimensional analysis (FIG. 8), it was noted that the charge and size of EPO derived from the transgenic mouse was more heterogeneous than those in the blood sample.

Generally, the PI value tends to become low as the terminal of a protein contains many sugar chains such as sialic acid.

As shown in FIG. 8, recombinant hEPO produced by the milk of the transgenic mouse in lines 6 and 37 is more acidic due to sialic acid-rich structure, compared to sera in control and patients with renal failure. By contrast, the blood collected from a control and patients with renal failure is asialic.

The sugar chain, such as sialic acid, affects the structure and function of a protein.

Sialic EPO has a physiological activity as it acts on the precursor of red blood cell, while asialic EPO is excreted into urine by binding to liver receptor (Parekh R B, et al. (1989) N-glycosylation and in vitro enzymatic activity of human recombinant tissue plasminogen activator expressed in Chinese hamster ovary cells and a murine cell line. Biochemistry 28: 7670-7679; Tam R C, et al. (1991) Comparisons of human, rat and mouse erythropoietins by isoelectric focusing: differences between serum and urinary erythropoietins. Br J Haematol 79: 504-511).

Therefore, hEPO produced by inventive EPO-expressing transgenic mouse is expected to exhibit a physiological activity by acting on the precursor of red blood cell.

5) Separation of hEPO from Milk of Transgenic Mouse

3 mL of milk, which was collected from inventive transgenic mouse, was suspended in 20 mL of 10 mM Tris buffer (pH 6.8) and filtered by a membrane filter. Using 1,000 mL of 10 mM Tris buffer (pH 6.8), the filtrate was dialyzed two times at 4° C. overnight. The dialyzed suspension was cooled and centrifuged at 4,000 rpm for 30 minutes to remove a precipitate.

About 20 mL of the supernatant was filtered by a pM-100 membrane filter

(Amicon) and then, the resulting filtrate was suspended in 300 mL of 10 mM Tris buffer (pH 6.8). At the flow rate of 3 mL/min, the suspension was injected to DEAE sephadex column (2φ×15 cm, bed volume 40) which was previously equilibrated with 10 mM Tris buffer (pH 6.8). The column was washed with 500 mL of 10 mM Tris buffer (pH 6.8) for gradient elution (each partition size: 3 mL, flow rate: 21 mL/h) using 10 mM Tris buffer (from 0 to 325 mM NaCl).

All of the eluted fractions containing EPO, so collected, were filtered by pM-10 membrane, washed with 10 mM Tris buffer (pH 7.2) containing 0.15M NaCl three times and concentrated to 3 mL. The solution was injected to Sephadex G-100 column (2F×100 cm) which was previously equilibrated with 10 mM Tris buffer (pH 7.2) and dissolved at the flow rate of 24 mL/h and fraction size of 3 m.

All of the eluted fractions containing EPO were recollected, filtered by pM-10 membrane and diluted with 10 mM Tris buffer (pH 7.5) containing 0.15M NaCl. The following immunopurification was conducted using the diluted product.

\hat{1} To Separate IgG from the Diluted Product, Ascitic Fluid was Collected for Purification using Protein-G Agarose Affinity System.

The ascitic fluid diluted in a 5-fold combined buffer (0.1M NaH₂PO₄, 0.15M NaCl, 5 mM EDTA, pH 7.0) was allowed to flow into Protein-A agarose column slowly and washed with the same buffer. The ascitic fluid, so washed, was eluted with elution buffer (0.1M glycine/HCl, 0.01% sodium azide, pH 2.7). The eluate was eluted into a tube containing the same amount of neutral buffer (1M Tris, 0.01% sodium azide, pH 9). These processes were conducted using 12% SDS-PAGE (sodium dodecyl sulfate-polyacrylamide gel electrophoresis) and identified by Coomassie Brilliant Blue, CBB). This antibody solution was dialyzed in 3L of PBS solution 6 times for 3 days, followed by quantitation for actual use.

\hat{2} Preparation of Immunoaffinity Column

50 mg of IgG, so purified, was dialyzed with carbonate hydrogen buffer (0.1M NaHCO₃, 0.5M NaCl, pH 8.3) at 4° C. overnight. 3 g of Sepharose 4B (Pharmacia), which was activated by CNBr, was washed with 1 mM hydrochloric acid several times. About 12 mL of gel poured into a column and washed with carbonate hydrogen buffer for equilibrium. Then, IgG was added to the column and stirred slowly overnight. The resulting solution was stirred with 1M ethanolamine solution (pH 8.8) at room temperature for 3 hours, and unreacted active functional groups were neutralized. Finally, the solution was washed with 30 mL of carbonate buffer three times, followed by once washing with 30 mL of 0.1M glycine-hydrochloric acid solution (pH 2.5). PBS buffer containing 0.2% NaN₃ was added to the washing solution and stored at 4° C. before use.

\hat{3} Immunopurification

A column was pretreated with 10 mM Tris buffer containing 0.15M NaCl (pH 7.5) for equilibrium. 20 mL of diluted sample solution were stirred gently at 4° C. overnight and absorbed to a gel. After the gel was poured into the column, the column was also pretreated with a buffer and washed with 0.1M acetate buffer (pH 4.5) at the flow rate of 60 mL/h. The EPO bound to the gel was eluted with 30 mL of 0.1M glycine-hydrochloric acid solution (pH 2.5). 1M Tris solution (pH 9) was immediately added to the eluted fractions and titrated up to pH 7.5 within a short time. Thereafter, the eluted fractions were filtered by pM-10 membrane (Amicon) to obtain hEPO.

### 6) Structural Analysis of Oligosaccharides in Erythropoietin

The oligosaccharide structure of hEPO identified in the milk of inventive transgenic mouse was analyzed by HPLC, using bovine serum fetuin (Glycosciences) and commercially available epoetin a (LG Life Sciences) as glycoprotein control.

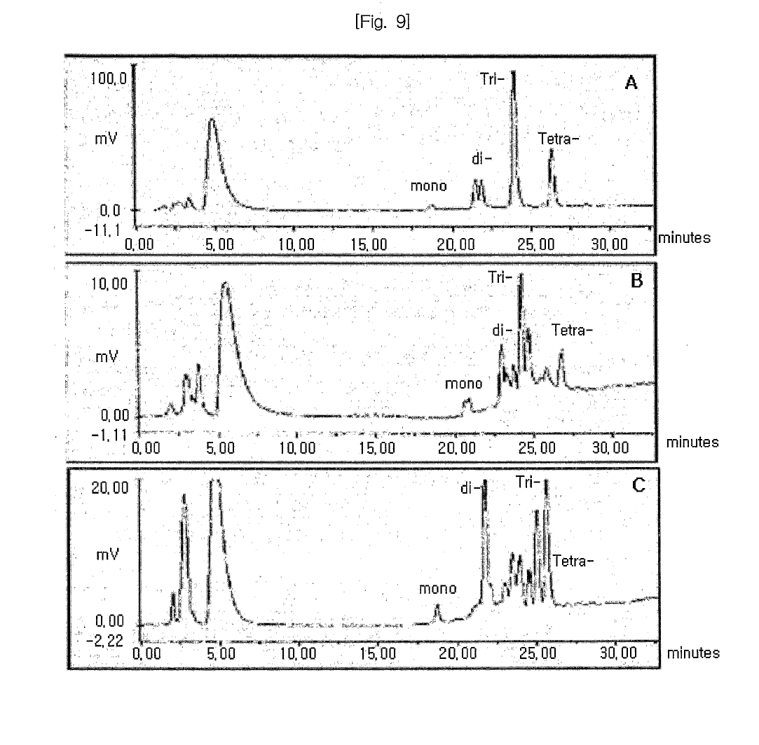

As shown in FIG. 9, A is a bovine serum fetuin, B is a transgenic mouse-derived hEPO, and C is epoetin α. It was confirmed that the transgenic mouse milk-derived EPO has similar mono-, di- and tri-acidic oligosaccharides to those of CHO cells-derived epoetin a, while the former has more tetra-acidic oligosaccharides than the latter.

This demonstrates that when EPO was expressed by glycosylation enzyme in the transgenic mouse, post-translational modification was normally made available, suggesting that EPO produced from the mammary gland could be easily used as a pharmaceutical product (FIG. 4).

TABLE 4
N-linked charge analysis of EPO in human serum and in milk of transgenic
mouse

| | Natural | Mono-acidic | Di-acidic | Tri-acidic | Tetra-acidic | |
|---|---|---|---|---|---|---|
| Source | glycan % | glycan % | glycan % | glycan % | glycan % | Comments |
| Human | 20 | 9 | 48 | 23 | 0 | Venke Kibeli et al. |
| serum EPO | | | | | | (2001) |
| | | | | | | BLOOD, 98(13) |
| | | | | | | 3626-3634 |
| Milk-derived | nd | 3.82 ± 0.2 | 26.75 ± 1.0 | 40.05 ± 1.9 | 29.38 ± 3.12^{a} | |
| rhEPO | | | | | | |
| Epoetin α | nd | 2.73± | 36.79 ± 0.4 | 40.23 ± 0.7 | 20.60 ± 0.17^{b} | |

Quantitative data were obtained from experiments performed in triplicate, and presented as mean ± S.D. Rows of different superscripts within a column are significantly different (P < 0.05).
nd represents ‘not detectable’

7) hEPO Activity in the Milk of Transgenic Mouse
7-A) In vitro hEPO Activity in the Milk of Transgenic Mouse

The following tests were conducted to investigate in vitro hEPO activity in the milk of inventive transgenic mouse.

EPO has been shown to activate STATS, transcription factor, by binding to EPO receptor (EPOR). With this in mind, we constructed BCL-XL luciferase expression vector containing the STATS to express EPOR in MCF-7 cell line (human breast cancer cell line) in a stable manner.

The MCF-7 cells (2×10⁵/35 mm plate) were treated with milk proteins (0 ng, 10 ng, 100 ng, 1 μg) containing rhEPO of transgenic mouse for 16 hours.

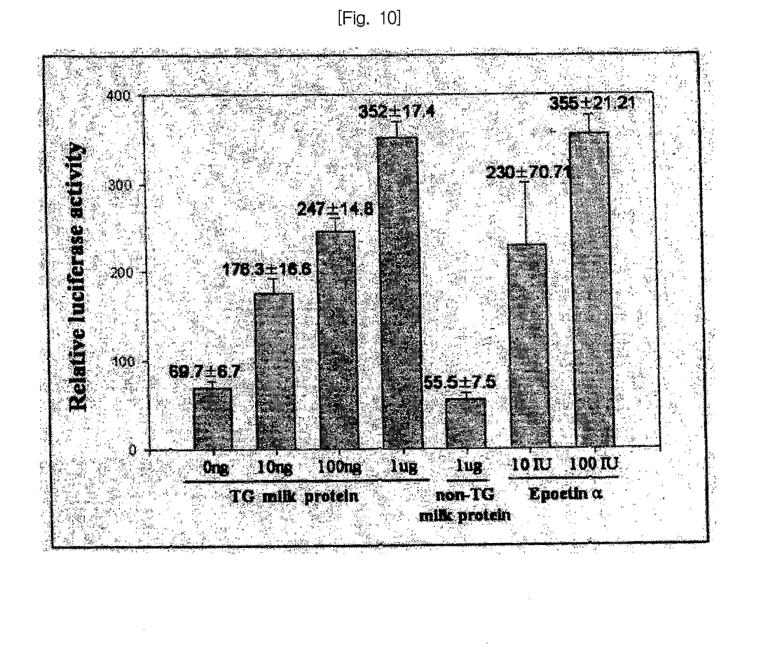

In negative control, the MCF-7 cells (2×10⁵/35 mm plate) were treated with 1 μg of general milk proteins. The cells were treated with Epoetin alpha (10 IU, 100 IU) as positive control. After 16 hours, relative activities of luciferase were measured using microplate luminometer (Perkin Elmer), as shown in FIG. 10.

From FIG. 10, it was noted that when the MCF-7 cells were treated with different concentrations of EPO, derived from the milk of transgenic mouse, increased luciferase activities were observed in a dose-dependent fashion.

As such increased luciferase activities was also observed by treatment of Epoetin alpha, we noted better in vitro activity of hEPO derived from the milk of inventive transgenic mouse.

7-B) In vivo Physiological Activity of EPO in the Milk of Transgenic Mouse

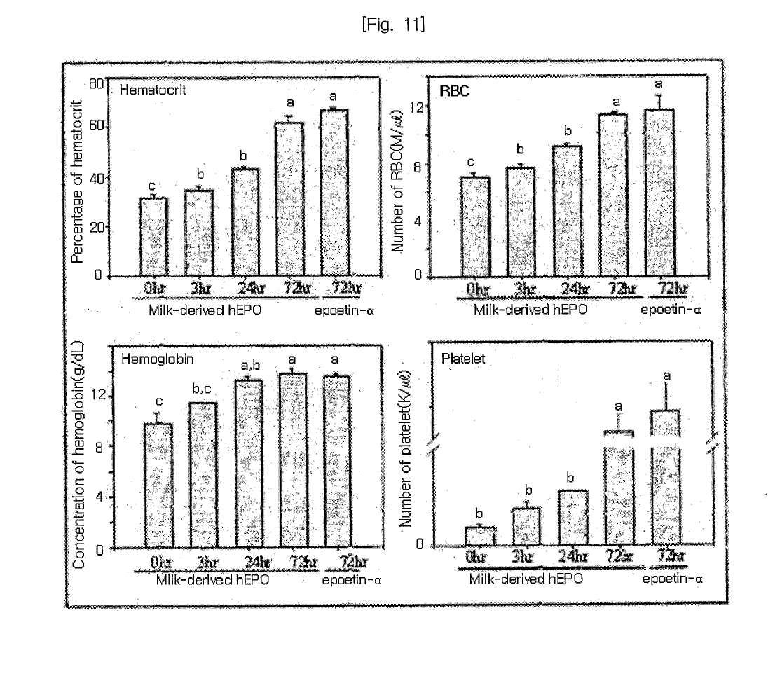

To investigate in vivo physiological activity of EPO in the milk of inventive transgenic mouse, EPO derived from the milk of transgenic mouse was injected to control mice (200 ng/kg, once intravenous injection) to collect each blood sample at different time intervals. The blood test results were shown in FIG. 11.

As shown in FIG. 11, the increased amounts of platelet, RBC, hemoglobin and hematocrit were observed in a time-dependent fashion, when EPO derived from the milk of inventive transgenic animal was added to control mouse.

As such increased physiological activities was also observed by treatment of Epoetin alpha, we noted better in vivo activity of EPO derived from the milk of inventive transgenic mouse.

### Example 3

### Production of Transgenic Pig using Somatic Cells through Introduction of Inventive EPO Expression Vector

Transgenic pig was produced using somatic cells through introduction of inventive expression vector pBC1/hEPO/NEO of Example 1.

### A) Preparation of Culture Media

NCSU 23 media was intended for use in in vitro maturation of follicular eggs. 1L of water from Baxter (Baxter Healthcare Co., U.S.A.) was filtered by a 0.2 μm filter (Gelman Sci., U.S.A.) and while pH was adjusted at 7.2-7.3, the filtrates were injected to 50 mL of tissue culture flask (Falcon, U.S.A.) by 45 mL each and stored at 4° C. for about 2-week use.

In vitro maturation medium was prepared using NCSU 23 with 10% porcine follicular fluid, 0.1 mg/mL cysteine, 0.01 μg/mL EGF, 10 IU/mL eCG and 10 IU/mL hCG. In vitro culture media was prepared using NCSU 23 with 0.4% BSA.

Porcine follicular fluid was collected from ovarian follicle (2-7 mm in diameter), centrifuged at 1,900×g three times, filtered by a 0.2 μm filter and stored at −20° C. before use.

### B) Collection of Porcine Follicular Oocytes

Ovaries of prepubertal gilts using this Example were enucleated immediately after slaughter at a local slaughterhouse, transferred to a thermos bottle containing physiological saline (30-35° C..) supplemented with penicillin G (100 units/mL) and streptomycin (100 μg/mL) and delivered to a laboratory within 3 or 4 hours. Prior to immature follicular oocytes, fatty and connective tissues around the ovaries were removed and washed with physiological saline 3 or 4 times. Oocytes were aspirated from cumulus-oocyte complexes (2-7 mm in diameter) using a 20 mL injector with 18-G needle.

The medium used for collection of oocytes was TALP-HEPES with 0.1 mg/mL PVA (Prather, R. S., et al. 1995. In vitro development of embryos from sinclair miniature pigs: A preliminary report. Theriogenology, 43:1001-1007).

Aspirated porcine follicular fluid was allowed to stand for 5-10 minutes.

Precipitated bottom solution was aspirated by a 5 mL pipette and placed in 60-mm culture dishes.

Oocytes were collected under an inverted microscope 40×multitude (Olympus Co., Japan) and selected by washing with in vitro maturation NCSU 23 4-5 times. Only oocytes with at least two-layer oocyte and excellently homogenous cytoplasm were selected for use of the following example.

C) In vitro Maturation of Porcine Follicular Oocytes

500 μL of porcine oocytes for in vitro maturation were placed in 4-well dishes (Nunc, Denmark) and cultured for 18 hours to induce the equilibrium.

100-150 oocytes with at least two-layer oocyte and excellently homogenous cytoplasm were placed in an in vitro maturation medium supplemented with hormone and cultured in a humidified (98-99%) 39° C. CO₂ incubator with 5% CO₂ for 20-22 hours. These oocytes were cultured in a hormone-free medium for 20-22 hours. Oocytes were cultured for a total of 40-44 hours.

D) Construction of Vector for Introduction and Targeting of hEPO-Expressing Gene

Based on the method described in Example 1, pBC1/hEPO/NEO expression vector expressing hEPO was constructed.

### E) Preparation of Somatic Donor Cells and Establishment of Cell Line

Porcine fetus in a 30-day gestation period was collected and used for this experiment.

Except for head, extremities and gut, all of the remaining tissue pieces were minced and placed in D-PBS supplemented with 0.05% trypsin (Gibco, USA) and EDTA (Sigma, USA) for 3 minutes. These pieces were centrifuged to remove trypsin and EDTA.

Isolated cells were placed in DMEM supplemented with 10% fetal bovine serum (FBS), plated in a 25 cm³ flask (Falcon, USA) and cultured at CO₂ incubator.

After a 12-hour culture, tissue pieces which were not settled on the bottom were removed. Cultures were replated with a fresh DMEM+10% FBS and fed for 3-5 days.

Donor cells showing more than 80% in growth in flask were treated with trypsin (0.05%) and EDTA for floating. Cells were then passaged 10 times by dividing into ⅓-¼.

Subcultured donor cells were frozen in DMEM supplemented with 10% DMSO and stored. For nuclear transfer, donor cells were thawed in water at 38-39° C. to remove a cryoprotectant. Cells were passaged one time in DMEM supplemented with 10% FBS. Cells were cultured in confluency for 2-3 days to harvest monolayers. The donor cells for this experiment were cells of G0 or G1 phase in cell-division cycle.

F) Introduction of Foreign Gene to Established Somatic Cells

pBC1/hEPO/NEO DNA, which was prepared by Example 1 and stored at −20° C. was thawed. Effectene transfection reagent (Qiagen), which was refrigerated, was also used. The two materials were placed in each tube whose DNA level was marked at 2 μg/mL, while avoiding their mixing. 8 μL of enhancer in Qiagen kit was slowly added to each tube.

Buffer EC was added to ensure that the total amount (DNA+enhancer+buffer EC) became 150 μL, vortexed for 1 second and stored at RT for 2-5 minutes. Each 25 μL of Effectene was added to the DNA-enhancer complex, followed by pipetting to place at RT for 5-10 minutes.

Further, fibroblasts which were prepared in Section E and grown to 50 to 80% confluency in the culture dish were washed with D-PBS 2 or 3 times for 10 minutes, plated with each 4 mL of FBS-free DMEM, and placed in an incubator.

After 10 minutes, FBS-free DMEM was added to each tube by 1 mL and mixed well by pipetting. Cells were placed in a dish containing 4 mL of fibroblast and DMEM and after transfection, cells were again left in an incubator for 12-18 hours. Thereafter, cells were washed with D-PBS, which was replaced by DMEM with 10% FBS-added.

### G) Selection of EPO-Introduced Somatic Cell Line EPO, Subculture and Freezing Preservation

72 hours after transfection date from the above paragraph F), neomycin-resistant cells were selected for about 2 weeks using 600-800 μg/mL of G418. Selected fibroblast cells were settled on the bottom for subculture.

More specifically, a medium was removed from a bottle of cells to be separated.

1.5 mL of 0.25% trypsin+EDTA was added to the bottle and left in an incubator for about 3-5 minutes. When about 70% of cells were distant under the microscope, cells were removed from the incubator and separated using a transfer pipette. Cells were transferred to a 15 mL tube containing 10 mL of D-PBS and centrifuged at 1500 rpm for 3 minutes. After removal of supernatants, about 3 mL of 10% FBS+DMEM+G418 was added to a pellet for sufficient dispersion. The dispersed pellet was put into a bottle and an incubator for culture.

For freezing preservation of cells, cells were centrifuged to obtain a pellet. 1 mL of DMSO was added to the pellet for sufficient dispersion. The pellet was put into a cryotube, stored at −70° C.. deep freezer for 24 hours and restored at −196° C. liquid nitrogen tank.

The porcine somatic cells containing pBC1/hEPO/NEO of the present invention was deposited to the Korea Research Institute of Bioscience and Biotechnology's

Biology Resource Center under the accession number KCTC 11160BP on Jul. 26, 2007.

### H) Preparation of Freezing Somatic Donor Cells for Nuclear Transfer

1. Thawing: Cells were Removed from a Refrigerator and Thawed at 37° C. Water Bath.

Thawed cells were transferred to a 15 mL tube containing 10 mL of D-PBS and centrifuged at 1500 rpm for 3 minutes. After removal of supernatants, about 3 mL of 10% FBS+DMEM+G418 was added to a pellet for sufficient dispersion. The dispersed pellet was put into a bottle and an incubator for culture.

2. Preparation of Donor Nuclei: Donor Cells should be Usually Prepared in a 4-Well Dish.

To separate cells, a medium was removed and then 200 μL of trypsin+EDTA was plated in a dish and left in an incubator for about 3-5 minutes. When about 70% of cells were distant under the microscope, cells were removed from the incubator and separated using a 200 μL pipette. Cells were placed in a dish containing about 3 mL of a medium for donor cells and allowed to disperse. Cells were stored in an incubator before use.

### I) Nuclear Transfer

For nuclear transfer, each pipettes for holding, enucleation and injection were prepared, using a capillary tube (1 mm in diameter; Narishige, Japan). The outer meter of a holding pipette was 150-180 μm, while those of enucleation and injection pipettes were adjusted at 30-40 μm. Manufactured pipettes were treated PVP coating before use.

In vitro matured recipient cytoplasm was placed in D-PBS supplemented with 0.1% hyaluronidase (Sigma, USA) for removal of cumulus cells and washed with PVA-TALP-HEPES with 3-4 times.

Only oocytes with excellent cytoplasm and a visible first polar body were selected in a medium supplemented with 0.05 M sucrose (Sigma, USA) and 0.4% BSA.

Enucleation was accomplished by aspirating 30% of cytoplasm from droplets of NCSU-23 medium with 0.4% BSA, containing 7.5 μg/mL cytochalasin B (Sigma, USA) and 0.05 M sucrose.

The enucleated oocytes were loaded with donor cells whose cycle was induced to G0 or G1 phase in a cytoplasm-free space so that cytoplasm could be attached to oocytes. The oocytes containing donor cells were treated with IVC medium ((NCSU-23 with 0.4% BSA) prior to electric fusion.

### J) Fusion of Reconstructed Embryo and Activation of Oocytes

The fusion between donor cells of cloned embryo and cytoplasm was performed using an electro-cell manipulator (BTX, USA). Reconstructed embryos were preincubated in activation medium, compring 0.28 M mannitol solution (Sigma, USA) supplemented with 0.1 mM CaCl₂ (Sigma, USA) and 0.1 mM MgCl₂ (Sigma, USA). The equilibrium was conducted in the medium for 2-3 minutes.

Electrical stimulation was delivered with the electro-cell manipulator to a chamber with two parallel wire electrodes. The reconstructed oocygtes and cytoplasm were pointed for anode (+) and cathode (−), respectively. Oocytes were exposed to an electrical pulse at 150V, followed by a 50 μsec 2 pulses. Following somatic cell injections, oocytes were either activated and then cultured in a medium containing cytochalasin B for 4 hours or were in vitro culture in a medium with 0.4% BSA.

### K) Preparation of Recipient Pigs

To transplant cloned embryos, PGF2α was intramuscularly administered to pregnant recipients of 30-40 days to induce abortion. 24 hours later, PGF2α and PMSG were simultaneously administered to the recipients. 72 hours later, hCG was intramuscularly administered to the pregnant mothers to induce superovulation. 48 hours later, cloned embryos were transplanted to the recipients.

### L) Embryo Transfer and Pregnancy Diagnosis

To produce cloned pigs, reconstructed embryos were surgically transferred into synchronized foster mothers. Ketamine and Rompun were administered to ear veins of hormone-treated synchronized recipients to induce general anesthesia. A razor was used to remove hairs of anesthetized recipients along the wide length a median line. After incision of the disinfected median line by about 10-15 cm, uterus was exposed to confirm corpus hemorrhagicum at the surface of the ovary or oocytes. Then, cloned embryos were injected to ampulla of urterine tube. To confirm development of cloned embryos in some recipients, abdomen was surgically incised 7 days after embryo transfer to assess embryonic development. An ultrasound scanner was also used to check pregnancies 27-30 days after embryo transfer. PCR and genomic Southern blot were performed to confirm transgenic porcinelets expressing mammary gland-specific EPO.

### Advantageous Effect

The inventive hEPO-expressing transgenic animals express a mammary gland-specific EPO at an extremely higher concentration than the convention method.

Further, hEPO produced from inventive transgenic animals shows better stability and superior physiological activity than those of the same kind of commercially available protein.

Further, hEPO produced from inventive transgenic animals increments the levels of platelet, erythrocyte, hemoglobin and hematocrit in the blood during administration.

Therefore, the inventive hEPO-expressing vectors and the transgenic animals can be effectively used for production of EPO showing a superior physiological activity than the existing EPO.

1. A pBC1-hEPO-WPRE vector comprising;
a DNA sequence encoding beta-casein promoter as a mammary gland-specific promoter;
a DNA sequence encoding human erythropoietin (hEPO) at 3′ end of the promoter; and
a DNA sequence encoding woodchuck hepatitis virus post-transcriptional regulatory element (WPRE) at 3′ end of the hEPO gene.

2. The vector of claim 1, wherein the DNA sequence encoding human hEPO is SEQ ID NO: 1.

3. The vector according to of claim 1, wherein the DNA sequence encoding beta-casein promoter is SEQ ID NO:2.

4. The vector of claim 1, wherein the DNA sequence encoding WPRE is SEQ ID NO:3.

5. A pBC1/hEPO/NEO vector, further comprising a neomycin-resistant gene as a selectable marker in the vector of claim 1.

6. The vector of claim 5, wherein the DNA sequence encoding the neomycin-resistant gene is SEQ ID NO:4.

7. The vector of claim 6, which is deposited with the Korea Research Institute of Bioscience and Biotechnology's Biology Resource Center under Accession Number KCTC 11159BP.

8. Somatic cells of a non-human animal that are transformed by introduction of the expression vector of claims 1.

9. The somatic cells of claim 8, wherein the cells:
are porcine somatic cells;
harbor pBC1/hEPO/NEO; and
are deposited with the Korea Research Institute of Bioscience and Biotechnology's Biology Resource Center under Accession Number KCTC 11160BP.

10. An embryo that is produced by nuclear transfer of the somatic cells of a non-human animal, transformed by introduction of the expression vector of claim 1, into an enucleated egg of a non-human animal.

11. A transgenic non-human animal that is transformed by introduction of the expression vector of claim 1.

12. The transgenic animal of claim 11, wherein the animal is selected from the group consisting of pig, mouse, cow, goat, sheep, horse, and dog.

13. A method for producing human erythropoietin (hEPO), comprising isolating and purifying the expressed EPO from milk produced by the transgenic animal of claim 11.

14. The method of claim 13, wherein the animal is selected from the group consisting of pig, mouse, cow, goat, sheep, horse, and dog.
